(19) United States
(12) Patent Application Publication
(10) Pub. No.: US 2012/0232171 A1
(43) Pub. Date: Sep. 13, 2012

(54) Alkoxylated Cyclic Diamines And Use Thereof As Emulsion Breakers

(75) Inventors: Carsten Cohrs, Frankfurt (DE); Stefan Dilsky, Gerbrunn (DE); Dirk Leinweber, Kelkheim (DE); Michael Feustel, Koengernheim (DE)

(73) Assignee: CLARIANT FINANCE (BVI) LIMITED, Tortola (VG)

(21) Appl. No.: 13/497,933

(22) PCT Filed: Sep. 3, 2010
(86) PCT No.: PCT/EP2010/005429

§ 371 Date: Mar. 23, 2012

(30) Foreign Application Priority Data
Sep. 24, 2009 (DE) 10 2009 042 971.9

(51) Int. Cl. B01D 17/05; C07C 211/18

(52) U.S. Cl. 516/161; 564/453

(57) ABSTRACT

The invention relates to the use of alkoxylated cyclic diamines, the reactive groups of which are alkoxylated by means of at least one C₂ to C₄ alkylene oxide, and the average degree of alkoxylation of which is between 1 and 200 alkylene oxide units per reactive group, in amounts of 0.0001 to 5 wt % relative to the oil content of the emulsion to be broken, to break water-in-oil emulsions.

The present invention relates to the use of alkoxylated cyclic diamines for breaking water-oil emulsions, especially in crude-oil production, and to appropriate diamines.

Crude oil is recovered in the form of an emulsion with water. Before further processing the crude oil, these crude-oil emulsions have to be broken to separate them into the oil portion and the water portion. This is generally done using so-called petroleum emulsion breakers. Petroleum emulsion breakers are surface-active polymeric compounds capable of effectuating the requisite separation in the emulsion constituents within a short time.

It is mainly alkoxylated alkylphenol-formaldehyde resins, nonionic alkylene oxide block copolymers and also variants crosslinked with bisepoxides that are used as demulsifiers. Overviews are given by “Something Old, Something New: A Discussion about Demulsifiers”, T. G. Balson, pp. 226-238 in Proceedings in the Chemistry in the Oil Industry VIII Symposium, Nov. 3-5, 2003, Manchester, GB, and also “Crude-Oil Emulsions: A State-Of-The-Art Review”, S. Kokal, pp. 5-13, Society of Petroleum Engineers SPE 77497.

U.S. Pat. No. 4,032,514 discloses the use of alkylphenol-aldehyde resins for breaking petroleum emulsions. These resins are obtainable by condensing a para-alkylphenol with an aldehyde, usually formaldehyde.

Such resins are often used in alkoxylated form, as disclosed in DE-A-24 45 873 for example. For this purpose, the free phenolic OH groups are reacted with an alkylene oxide.

In addition to the free phenolic OH groups, free OH groups of alcohols or NH groups of amines can also be alkoxylated, as disclosed in U.S. Pat. No. 5,401,439 for example.

U.S. Pat. No. 4,321,146 and U.S. Pat. No. 5,445,765 disclose alkylene oxide block copolymers and alkoxylated polyethyleneimines respectively as further petroleum emulsion breakers.

Alkoxylated dendritic polyesters (dendrimers) are disclosed as biodegradable (OECD 306) petroleum emulsion breakers in DE-A-103 29 723. DE-A-103 25 198 likewise discloses breakers biodegradable to OECD 306. Alkoxylated, crosslinked polyglycerols are concerned here.

The disclosed breakers can be used as individual components or in mixtures with other emulsion breakers.

The different properties (e.g., asphaltene, paraffin and salt contents, chemical composition of the natural emulsifiers) and water fractions of various crude oils make it imperative to further develop the existing petroleum breakers. Particularly a low dosage rate and broad applicability of the petroleum breaker to be used is at the focus of economic and ecological concern as well as the higher effectivity sought.

It is an object of the present invention to develop novel petroleum breakers which are equivalent or superior to the existing petroleum breakers in performance, and can be used in even lower dosage.

Surprisingly, alkoxylated cyclic diamines, optionally after crosslinking with multifunctional glycide ethers, are found to give excellent performance as emulsion breakers at very low dosage.

The invention accordingly provides for the use of cyclic diamines whose reactive groups are alkoxylated with at least one C₂ to C₄ alkylene oxide, so that the average degree of alkoxylation is between 1 and 200 alkylene oxide units per reactive group, the average degree of alkoxylation here being the average number of alkoxy units which are attached to each reactive group, in amounts of 0.0001% to 5% by weight, based on the oil content of the emulsion to be broken, for breaking water-in-oil emulsions.

The present invention further provides a process for breaking a water-in-oil emulsion by adding to the emulsion from 0.0001% to 5% by weight, based on the oil content of the emulsion, of at least one cyclic diamine, the reactive groups of which are alkoxylated with at least one C₂ to C₄ alkylene oxide, so that the average degree of alkoxylation is between 1 and 200 alkoxy units per reactive group, the average degree of alkoxylation here being the average number of alkoxy units which is attached to each reactive group.

The invention further provides cyclic diamines whose reactive groups are alkoxylated with at least one C₂ to C₄ alkylene oxide, so that the average degree of alkoxylation is between 1 and 200 alkylene oxide units per reactive group and the average degree of alkoxylation here being the average number of alkoxy units which are attached to each reactive group.

In one preferable embodiment, the average degree of alkoxylation is 2 to 150 alkylene oxide units and more preferably 3 to 100 alkylene oxide units per reactive group. Reactive group refers to functional groups that have active H-atoms and thereby are accessible to alkoxylation.

The cyclic diamines used as an intermediate in the present invention are obtainable by reductive aminomethylation of cyclic diolefins as disclosed in EP-A-1 813 595 for example. It is preferable to use cyclic diamines of the general formula

H₂N—(CR¹R²)ₙ-Cyc-(CR³R⁴)ₘ—NH₂

where
Cyc represents an aliphatic mono-, di- or tricyclic unit containing altogether 4-20 carbon atoms,
R¹, R², R³ and R⁴ each independently represent H or methyl,
n represents a number from 0 to 3, and
m represents a number from 0 to 3.

In the aforementioned embodiment, Cyc can be a ring system without substituents, or Cyc can bear hydrocarbonaceous substituents having 1 to 6 carbon atoms. Examples of suitable hydrocarbonaceous substituents are methyl, ethyl, propyl or butyl, and also vinyl and allyl. The groups —(CR¹R²)ₙ—NH₂ and —(CR³R⁴)ₘ—NH₂ do not count as substituents within this meaning.

Cyc can be a ring system composed of carbon and hydrogen, it can also comprise heteroatoms as ring members. Nitrogen and oxygen are suitable heteroatoms. When Cyc contains nitrogen atoms, these are preferably tertiary-substituted.

Cyc preferably contains not more than 14 carbon atoms.

Examples of aliphatic cyclic units Cyc are cyclopentane, cyclohexane, cycloheptane, cyclooctane, pyrrolidine, piperidine, piperazine, decahydronaphthalene (=decalin), bicyclo[2.2.1]heptane (=norbonane), 2,6,6-trimethylbicyclo[3.1.1]heptane (=pinane), bicyclo[2.2.2]octane, bicyclo[3.2.1]octane, bicyclo[4.3.0]nonane and tricyclo[5.2.1.0^{2,6}]decane (=tetrahydrodicyclopentadiene).

Examples of diamines constructed from such cyclics are TCD-diamine, 3(4),7(8)-bis(aminomethyl)bicyclo[4.3.0]nonane, isophoronediamine, 1,8-diamino-p-menthane, 1,2-cyclohexanediamine, 1,3-cyclohexanediamine, 1,4-cyclohexanediamine or 1,4-bis(3-aminopropyl)piperazine.

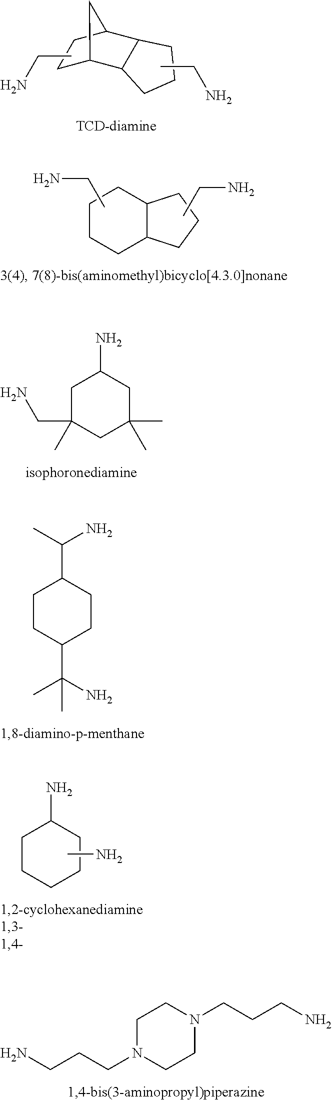

In one preferable embodiment, the alkoxylated cyclic diamines are subjected to crosslinking. Crosslinking can take place both before and after alkoxylation. It is thus possible for the unalkoxylated cyclic diamines which are useful as intermediates first to be crosslinked and then alkoxylated. It is also possible to crosslink the already alkoxylated cyclic diamines.

The following crosslinkers are particularly preferable for the crosslinking reaction: bisphenol A diglycidyl ether, butane-1,4-diol diglycidyl ether, hexane-1,6-diol diglycidyl ether, ethylene glycol diglycidyl ether, cyclohexanedimethanol diglycidyl ether, resorcinol diglycidyl ether, glycerol diglycidyl ether, glycerol triglycidyl ether, glycerol propoxylate triglycidyl ether, polyglycerol polyglycidyl ether, p-aminophenol triglycidyl ether, polyethylene glycol diglycidyl ether, polypropylene glycol diglycidyl ether, pentaerythritol tetraglycidyl ether, sorbitol polyglycidyl ether, trimethylolpropane triglycidyl ether, castor oil triglycidyl ether, diaminobiphenyl tetraglycidyl ether, neopentylglycol diglycidyl ether, but-2-ene-1,4-diol diglycidyl ether, perhydro bisphenol A diglycidyl ether.

In one preferable embodiment, the crosslinking step is carried out with 0.1-1.0 mol and more preferably 0.2-0.8 mol of the crosslinker, based on the cyclic diamine, before alkoxylation.

The crosslinked cyclic diamines obtained from this crosslinking step are subsequently alkoxylated with one or more C₂-C₄ alkoxides, preferably ethylene oxide (EO) or propylene oxide (PO), in this embodiment, wherein the alkylene oxide units form a random arrangement or, as in the case of a preferable embodiment, a block-type arrangement. The molar ratio of PO to EO is preferably between 100:1 and 1:100, more preferably between 60:1 and 1:60 and especially between 30:1 and 1:30.

In a further preferable embodiment, the crosslinking step is carried out after propoxylation and before subsequent ethoxylation, or after ethoxylation and subsequent propoxylation of cyclic diamines, using 0.5-10%, more preferably 0.8% to 8% and specifically 1-5% by weight of crosslinker, based on the alkoxylate. The ratio of PO to EO is preferably between 100:1 and 1:100, more preferably between 60:1 and 1:60 and especially between 30:1 and 1:30.

In a further preferable embodiment, the crosslinking step is carried out after alkoxylation of cyclic diamines using 0.5-10%, more preferably 0.8% to 8% and specifically 1-5% by weight of crosslinker, based on the alkoxylate. The ratio of PO to EO is preferably between 100:1 and 1:100, more preferably between 60:1 and 1:60 and especially between 30:1 and 1:30.

The alkoxylated cyclic diamines obtained after crosslinking and alkoxylation preferably have a molecular weight of 500 to 200 000 units and especially of 1000 to 100 000 units.

The alkoxylated cyclic diamines in a preferable embodiment have a water number of 10-26. The water number is a dimensionless number and is determined in accordance with DIN EN 12836.

The water number describes the hydrophilic-lipophilic balance (HLB) value of surface-active substances and is a measure of the water solubility of alkoxylated cyclic diamines. A high water number of more than 18 indicates good solubility in water, while a low water number of less than 13 indicates good solubility in oil. The water number depends on the ratio of EO groups to PO groups. Alkoxylated cyclic diamines of high water number lead to quicker breaking of emulsions, but produce separated-off water of comparatively great oil content. When the water number is small, breaking is slower but in turn the oil content of the removed water is lower.

A preferred aspect of the present invention is the use of alkoxylated crosslinked cyclic diamines as breakers for oil/water emulsions in petroleum production.

To be used as petroleum breakers, the alkoxylated crosslinked cyclic diamines are added to the water-in-oil emulsions, which is preferably done in solution. Paraffinic, aromatic or alcoholic solvents are preferable for the alkoxylated crosslinked cyclic diamines. The alkoxylated crosslinked cyclic diamines are used in amounts of 0.0001% to 5%, preferably 0.0005% to 2%, especially 0.0008% to 1% and specifically 0.001% to 0.1% by weight, based on the oil content of the emulsion to be broken.

### EXAMPLES

### General Prescription 1

Amine Crosslinking Before Alkoxylation 1 mol of diamine, 0.2-0.8 mol of crosslinker and alkaline catalyst (final alkali number 0.5-3.5 mg KOH/g) were mixed in a 500 ml three-neck flask equipped with contact thermometer, stirrer and reflux condenser. Under agitation, the reaction mixture was incrementally heated to 120° C. over 3 hours and then post-reacted at 120° C. Completeness of reaction (generally 3-4 hours) is determined by determining the epoxy number.

### General Prescription 2

Crosslinking the Alkoxylated Amine 1 mol of diamine, 1.0-5.0% by weight of crosslinker and alkaline catalyst (final alkali number 0.5 mg KOH/g) were mixed in a 500 ml three-neck flask equipped with contact thermometer, stirrer and reflux condenser. Under agitation, the reaction mixture was incrementally heated to 120° C. over 3 hours and then post-reacted at 120° C. Completeness of reaction (generally 6-8 hours) is determined by determining the epoxy number.

### General Prescription for Alkoxylation

### Ethoxylating Crosslinked Amine

The crosslinked diamines obtained according to the general crosslinking prescriptions 1 and 2, or propoxylates thereof, were introduced into a 1 l glass autoclave and adjusted with sodium methoxide solution to a final alkali number of about 2.5 mg of KOH/g of substance. The autoclave was inertized with nitrogen, pressure tested and then heated to 135° C. and the pressure in the autoclave was adjusted with nitrogen to about 0.8-1.0 bar. Thereafter, the desired quantity of ethylene oxide was metered in at not more than 140° C., while the pressure should not exceed 4.5 bar. On completion of the metered addition the reaction mixture is post-reacted at not more than 140° C. to constant pressure.

### Propoxylating Crosslinked Amine

The crosslinked diamines obtained according to the general crosslinking prescriptions 1 and 2, or ethoxylates thereof, were introduced into a 1 l glass autoclave and adjusted with sodium methoxide solution to a final alkali number of about 1.5 mg of KOH/g of substance. The autoclave was inertized with nitrogen, pressure tested and then heated to 125° C. and the pressure in the autoclave was adjusted with nitrogen to about 0.8-1.0 bar. Thereafter, the desired quantity of propylene oxide was metered in at not more than 130° C., while the pressure should not exceed 3.5 bar. On completion of the metered addition the reaction mixture is post-reacted at not more than 130° C. to constant pressure.

### Ethoxylating Uncrosslinked Amine

The diamines or their propoxylates were introduced into a 1 l glass autoclave and adjusted with sodium methoxide solution to a final alkali number of about 2.5 mg KOH/g of substance. The autoclave was inertized with nitrogen, pressure tested and then heated to 135° C. and the pressure in the autoclave was adjusted with nitrogen to about 0.8-1.0 bar. Thereafter, the desired quantity of ethylene oxide was metered in at not more than 140° C., while the pressure should not exceed 4.5 bar. On completion of the metered addition the reaction mixture is post-reacted at not more than 140° C. to constant pressure.

### Propoxylating Uncrosslinked Amine

The diamines or their ethoxylates were introduced into a 1 l glass autoclave and adjusted with sodium methoxide solution to a final alkali number of about 1.5 mg KOH/g of substance. The autoclave was inertized with nitrogen, pressure tested and then heated to 125° C. and the pressure in the autoclave was adjusted with nitrogen to about 0.8-1.0 bar. Thereafter, the desired quantity of propylene oxide was metered in at not more than 130° C., while the pressure should not exceed 3.5 bar. On completion of the metered addition the reaction mixture is post-reacted at not more than 130° C. to constant pressure.

TABLE 1
TCD-Diamine + butane-1,4-diol diglycidyl ether + PO + EO

| | | mol of | mol of | |
|---|---|---|---|---|
| | Molar ratio | PO per | EO per | |
| | of amine to | mol of | mol of | |
| Example | crosslinker | diamine | diamine | Water number |
| 1 | 1:0.5 | 26 | 0 | 11.6 |
| 2 | 1:0.5 | 26 | 7.5 | 17.5 |
| 3 | 1:0.5 | 26 | 11 | 19.7 |
| 4 | 1:0.5 | 26 | 12.5 | 20.4 |
| 5 | 1:0.5 | 26 | 14 | 21.3 |
| 6 | 1:0.5 | 26 | 16 | 22.6 |
| 7 | 1:0.5 | 31 | 0 | 11.4 |
| 8 | 1:0.5 | 31 | 9 | 17 |
| 9 | 1:0.5 | 31 | 12 | 19.2 |
| 10 | 1:0.5 | 31 | 14 | 20.5 |
| 11 | 1:0.5 | 31 | 16.5 | 21.4 |
| 12 | 1:0.5 | 31 | 18.5 | 22.2 |
| 13 | 1:0.5 | 36 | 0 | 10.5 |
| 14 | 1:0.5 | 36 | 8 | 15.2 |
| 15 | 1:0.5 | 36 | 11 | 17.8 |
| 16 | 1:0.5 | 36 | 14 | 18.7 |
| 17 | 1:0.5 | 36 | 16 | 19.5 |
| 18 | 1:0.5 | 36 | 19 | 21.8 |
| 19 | 1:0.5 | 42 | 0 | 10.5 |
| 20 | 1:0.5 | 42 | 8.5 | 15.1 |
| 21 | 1:0.5 | 42 | 14 | 17.8 |
| 22 | 1:0.5 | 42 | 18.5 | 20.4 |

TABLE 2
Isophoronediamine + butane-1,4-diol diglycidyl ether + PO + EO

| | | mol of | mol of | |
|---|---|---|---|---|
| | Molar ratio | PO per | EO per | |
| | of amine to | mol of | mol of | |
| Example | crosslinker | diamine | diamine | Water number |
| 23 | 1:0.5 | 20 | 0 | 13.1 |
| 24 | 1:0.5 | 20 | 6 | 17.5 |
| 25 | 1:0.5 | 20 | 8 | 18.1 |
| 26 | 1:0.5 | 20 | 10 | 21.1 |
| 27 | 1:0.5 | 39 | 0 | 13.2 |
| 28 | 1:0.5 | 39 | 12 | 14.7 |
| 29 | 1:0.5 | 39 | 18 | 16.3 |

TABLE 3
Isophoronediamine + bisphenol A diglycidyl ether + PO + EO

| | | mol of | mol of | |
|---|---|---|---|---|
| | Molar ratio | PO per | EO per | |
| | of amine to | mol of | mol of | |
| Example | crosslinker | diamine | diamine | Water number |
| 30 | 1:0.25 | 20 | 0 | 16.1 |
| 31 | 1:0.25 | 20 | 7 | 19.5 |
| 32 | 1:0.25 | 20 | 13 | 21.2 |
| 33 | 1:0.25 | 20 | 21 | 22.4 |

TABLE 4
TCD-Diamine + PO + bisphenol A diglycidyl ether + EO

| | mol of | | mol of | |
|---|---|---|---|---|
| | PO per | | EO per | |
| | mol of | % by weight of | mol of | |
| Example | diamine | crosslinker | diamine | Water number |
| 34 | 20 | 2.5 | 29 | 22.8 |
| 35 | 20 | 2.5 | 43 | 23.7 |
| 36 | 20 | 5 | 25.5 | 22.3 |
| 37 | 20 | 5 | 38.5 | 23.0 |
| 38 | 40 | 2.5 | 16.5 | 15.2 |
| 39 | 40 | 2.5 | 53.5 | 23.2 |
| 40 | 40 | 2.5 | 76 | 24 |
| 41 | 40 | 5 | 20.5 | 16.1 |
| 42 | 40 | 5 | 44.5 | 22.9 |
| 43 | 40 | 5 | 68 | 24.5 |

TABLE 5
TCD-Diamine + PO+ EO + bisphenol A diglycidyl ether

| | mol of | mol of | | |
|---|---|---|---|---|
| | PO per | EO per | | |
| | mol of | mol of | % by weight of | |
| Example | diamine | diamine | crosslinker | Water number |
| 44 | 40 | 22 | 0 | 18.1 |
| 45 | 40 | 44 | 0 | 21.6 |
| 46 | 40 | 63 | 0 | 23.7 |
| 47 | 40 | 22 | 2.5 | —* |
| 48 | 40 | 44 | 2.5 | —* |
| 49 | 40 | 63 | 2.5 | —* |
| 50 | 60 | 22.5 | 0 | 15.4 |
| 51 | 60 | 31.5 | 0 | 19.5 |
| 52 | 60 | 38 | 0 | 25.5 |
| 53 | 60 | 21.5 | 2.5 | —* |
| 54 | 60 | 31.5 | 2.5 | —* |
| 55 | 60 | 38 | 2.5 | —* |

*No water number determination was possible at final crosslinking

### Determination of Breaking Efficacy of Petroleum Emulsion Breakers

Emulsion breaker efficacy was determined by determining water separation from a crude-oil emulsion per unit time and also the dewatering of the oil. To this end, breaker glasses (conically tapered, graduated glass bottles closeable with a screw top lid) were each filled with 100 ml of the crude-oil emulsion, a defined amount of the emulsion breaker was in each case added with a micropipette just below the surface of the oil emulsion, and the breaker was mixed into the emulsion by intensive shaking. Thereafter, the breaker glasses were placed in a temperature control bath and water separation was tracked.

On completion of emulsion breaking, samples of the oil were taken from the top part of the breaker glass (top oil). A 15 ml centrifuge vial (graduated) is filled with 5 ml of Shellsol® A 150 ND and 10 ml of oil sample, the vial is shaken by hand to achieve commixing, and is then centrifuged at 1500 rpm for 5 minutes. After centrifuging, three phases are observed in the centrifuge vial: a clear aqueous phase, a brown emulsion phase and a black oily phase. The volumes read off for the aqueous and emulsion phases are multiplied by a factor of 10 and values thus determined are reported as % water and % emulsion. The remainder to 100% is the oily phase. Demulsification is particularly good when the sum total of % water and % emulsion is very small. Comparing two equal sum totals of % water and % emulsion, it is preferable for the % water fraction to be as large as possible. In this way, the novel breakers were assessed in terms of water separation and also oil dewatering. The quality of the water separated off was assessed by a practiced observer:
the entry “+” means that the water separated off is clear
the entry “◯” means that the water separated off is cloudy
the entry “−” means that the water separated off is nontransparent owing to oiling.

### Breaking Effect of Breakers Described

Origin of crude-oil emulsion: Hebertshausen, Germany
Water content of emulsion: 48%
Demulsifying temperature: 50° C.

Table 6 reports the efficacy of alkoxylated cyclic diamines as emulsion breakers compared with Dissolvan V 5252-1c. and Dissolvan V 5566-1c. (100 ppm) in terms of water separation in ml after the stated time.

TABLE 6
Breaking efficacy

| | | Water separation [ml] after | | | | | | | |
|---|---|---|---|---|---|---|---|---|---|
| | | stated time [min] | | | | | % | % | |
| Example | Product of example | 10 | 20 | 30 | 60 | 120 | water | emulsion | Water |
| 56 | 10 | 1 | 8 | 21 | 39 | 44 | 2 | 0 | + |
| 57 | 24 | 0.5 | 4 | 16 | 40 | 42 | 1 | 1 | ∘ |
| 58 | 25 | 4 | 17 | 28 | 40 | 42 | 1.5 | 1 | ∘ |
| 59 | 34 | 0.5 | 4 | 17 | 30 | 32 | 0.5 | 0.5 | + |
| 60 | 35 | 1.5 | 8 | 20 | 38 | 46 | 1 | 0 | + |
| 61 | 36 | 0.5 | 2 | 12 | 34 | 36 | 1 | 1 | + |
| 62 | 38 | 6 | 28 | 40 | 44 | 46 | 2 | 0 | + |
| 63 | 41 | 2 | 15 | 32 | 42 | 46 | 2 | 0 | + |
| 64 | 44 | 2 | 14 | 42 | 42 | 46 | 2.5 | 0 | + |
| 65 | 45 | 0.5 | 3 | 10 | 36 | 38 | 1 | 1 | ∘ |
| 66 | 47 | 3 | 28 | 43 | 45 | 46 | 2.5 | 0 | + |
| 67 | 48 | 0.5 | 2 | 6 | 34 | 36 | 0.5 | 2 | ∘ |
| 68 | 50 | 0.5 | 14 | 34 | 44 | 48 | 1 | 0 | + |
| 69 | 51 | 2 | 12 | 33 | 43 | 46 | 1 | 0 | + |
| 70 | 53 | 23 | 30 | 33 | 45 | 46 | 0.5 | 1.5 | + |
| 71 | 54 | 3 | 17 | 30 | 42 | 44 | 1.5 | 0.5 | + |
| 72 | Dissolvan ® | 1 | 5 | 33 | 40 | 44 | 2 | 1 | + |
| (comp) | V 5252-1c. | | | | | | | | |
| 73 | Dissolvan ® | 22 | 36 | 42 | 45 | 48 | 0.5 | 2.5 | ∘ |
| (comp) | V 5566-1c. | | | | | | | | |

What is claimed is:

1. The use of alkoxylated cyclic diamines whose reactive groups are alkoxylated with at least one C₂ to C₄ alkylene oxide and whose average degree of alkoxylation is between 1 and 200 alkylene oxide units per reactive group, in amounts of 0.0001% to 5% by weight, based on the oil content of the emulsion to be broken, for breaking water-in-oil emulsions.

2. The use according to claim 1 wherein the alkoxylated cyclic diamines are in a crosslinked state.

3. The use according to claim 1 and/or 2 utilizing a cyclic diamine as per formula (I),
H₂N—(CR¹R²)ₙ-Cyc-(CR³R⁴)ₘ—NH₂ (1)
where
Cyc represents an aliphatic mono-, di- or tricyclic unit containing altogether 4-20 carbon atoms,
R¹, R², R³ and R⁴ each independently represent H or methyl,
n represents a number from 0 to 3, and
m represents a number from 0 to 3.

4. The use according to claim 2 and/or 3 wherein the crosslinking is effected with multifunctional, glycidyl ethers before the alkoxylation and the molar ratio of multifunctional glycidyl ether to cyclic diamine is 0.2-0.8.

5. The use according to claim 2 and/or 3 wherein the crosslinking is effected with multifunctional glycidyl ethers at blockwise alkoxylation between the individual blocks.

6. The use according to claim 2 and/or 3 wherein the crosslinking is effected with multifunctional glycidyl ethers after the alkoxylation.

7. The use according to one or more of claims 5 and/or 6 wherein the multifunctional glycidyl ether is used at 1-5% by weight, based on the alkoxylated diamine.

8. The use according to one or more of claims 2-7 wherein the crosslinker is selected from bisphenol A diglycidyl ether, butane-1,4-diol diglycidyl ether, hexane-1,6-diol diglycidyl ether, ethylene glycol diglycidyl ether, cyclohexanedimethanol diglycidyl ether, resorcinol diglycidyl ether, glycerol diglycidyl ether, glycerol triglycidyl ether, glycerol propoxylate triglycidyl ether, polyglycerol polyglycidyl ether, p-aminophenol triglycidyl ether, polyethylene glycol diglycidyl ether, polypropylene glycol diglycidyl ether, pentaerythritol tetraglycidyl ether, sorbitol polyglycidyl ether, trimethylolpropane triglycidyl ether, castor oil triglycidyl ether, diaminobiphenyl tetraglycidyl ether, neopentylglycol diglycidyl ether, but-2-ene-1,4-diol diglycidyl ether, perhydro bisphenol A diglycidyl ether.

9. The use according to one or more of claims 1 to 8 wherein propylene oxide and ethylene oxide are used as alkylene oxides and the ratio of propylene oxide units to ethylene oxide units in the alkoxylated cyclic diamine is between 30:1 and 1:30.

10. The use according to one or more of claims 1 to 9 wherein the average degree of alkoxylation per reactive group is between 3 and 100.

11. The use according to one or more of claims 1 to 10 wherein the alkoxylated cyclic diamines have a molecular weight of 1000 to 100 000 units.

12. The use according to one or more of claims 1 to 5 and 7 to 11 wherein the alkoxylated cyclic diamines have a water number of 10 to 26.

13. An alkoxylated cyclic diamine whose reactive groups are alkoxylated with at least one C₂ to C₄ alkylene oxide and whose average degree of alkoxylation is between 3 and 100 alkylene oxide units per reactive group and which has a number average molecular weight of 1000 to 100 000 g/mol.

14. The alkoxylated cyclic diamine according to claim 13 in a crosslinked state.

15. The alkoxylated crosslinked cyclic diamine according to claim 14 wherein the diamine conforms to formula (I)
H₂N—(CR¹R²)ₙ-Cyc-(CR³R⁴)ₘ—NH₂ (1)
where
Cyc represents an aliphatic mono-, di- or tricyclic unit containing altogether 4-20 carbon atoms,
R¹, R², R³ and R⁴ each independently represent H or methyl,
n represents a number from 0 to 3, and
m represents a number from 0 to 3.

16. The alkoxylated crosslinked cyclic diamine according to claim 14 and/or 15 wherein the crosslinker is selected from bisphenol A diglycidyl ether, butane-1,4-diol diglycidyl ether, hexane-1,6-diol diglycidyl ether, ethylene glycol diglycidyl ether, cyclohexanedimethanol diglycidyl ether, resorcinol diglycidyl ether, glycerol diglycidyl ether, glycerol triglycidyl ether, glycerol propoxylate triglycidyl ether, polyglycerol polyglycidyl ether, p-aminophenol triglycidyl ether, polyethylene glycol diglycidyl ether, polypropylene glycol diglycidyl ether, pentaerythritol tetraglycidyl ether, sorbitol polyglycidyl ether, trimethylolpropane triglycidyl ether, castor oil triglycidyl ether, diaminobiphenyl tetraglycidyl ether, neopentylglycol diglycidyl ether, but-2-ene-1,4-diol diglycidyl ether, perhydro bisphenol A diglycidyl ether.

17. A process for breaking a water-in-oil emulsion by adding to the emulsion from 0.0001% to 5% by weight, based on the weight of the emulsion, of at least one alkoxylated cyclic diamine which has a number average molecular weight of 1000 to 100 000 g/mol, the reactive groups of which are alkoxylated with at least one C₂ to C₄ alkylene oxide, so that the average degree of alkoxylation is 3 to 100 alkoxy units per reactive group.
